(19) United States
(12) Patent Application Publication
(10) Pub. No.: US 2012/0213713 A1
(43) Pub. Date: Aug. 23, 2012

(54) Compositions of omega fatty acids for the prevention and treatment of dental caries resulting from oral infections

(75) Inventors: Chifu Huang, Lexington, KY (US); Jeffrey Lee Ebersole, Lexington, KY (US)

(73) Assignee: Chifu Huang, Lexington, KY (US)

(21) Appl. No.: 13/031,165

(22) Filed: Feb. 18, 2011

(51) Int. Cl. A61K 31/202; A61K 31/355; A61P 31/04; A61K 9/68; A61K 8/92; A61Q 11/00; A61K 31/341; A61K 31/59

(52) U.S. Cl. 424/48; 514/560; 514/474; 514/458; 514/167; 424/49

(57) ABSTRACT

The present invention discloses compositions of omega polyunsaturated fatty acids for controlling and preventing dental caries resulting from oral infections. The compositions of fatty acids include omega-3 fatty acids, omega-6 fatty acids, and other fatty acids, for their synergistic bioactivity against cariogenic pathogens. The application of this composition can be used for the preparation of drugs, nutritional supplements, food and daily necessities with oral health care and therapeutic effects.

### FIELD OF THE INVENTION

The present invention relates to compositions of fatty acids, which include omega-3 fatty acids, omega-6 fatty acids, and other fatty acids, for synergistic bioactivity. Specifically, the combinations of omega-3 fatty acids, eicosapentaenoic acid (EPA) and α-linolenic acid (ALA), omega-6 fatty acids (GLA), hexanoic acid (C6), myristic acid (C14), palmitic acid (C16), and palmitoleic acid (C16), and their ester derivatives, have strong anti-microbial activity against cariogenic pathogens, and the application of these compositions in products for the prevention and treatment of dental caries.

### BACKGROUND OF THE INVENTION

Omega-3 fatty acids include eicosapentaenoic acid (EPA) and α-linolenic acid (ALA) and others, belonging to n-3 unsaturated fatty acids (also known as the ω-3 unsaturated fatty acids or omega-3 fatty acid) family. Their common feature, the first double bond in the carbon chain located at third position, has played an important role in physiological functions. Other omega-6 fatty acids, such as y-linolenic acid (GLA), also play important physiological functions.

For example, omega-3 fatty acids, eicosapentaenoic acid (EPA) and α-linolenic acid (ALA), have been suggested to contribute a range of health benefits, primarily related to activities in minimizing inflammation, and as antioxidants. It is reported that omega-3 fatty acids can serve as precursors to groups of eicosanoids metabolites which can reduce inflammation. While omega-3 fatty acids have been found to reduce oral inflammation and may have potential benefits as a host modulatory agent in the prevention and/or adjunctive management of periodontitis, negligible data is available elucidating the potential for these agents to affect dental caries.

A recent world-wide survey reported that 80% of the world population in both developed and undeveloped countries suffer some level of dental caries. Dental caries is the predominant cause of tooth loss in children and young adults, as well as an increasing presence in the elderly (ie. root caries) and remains the most prevalent of all chronic diseases among children in the US, 5× more common than asthma and 7× more common than hay fever. Nearly 20% of all children by 2-4 years of age, >50% by 6-8 years, and over ¾ of adolescents by age 17 are affected. There is a great need for cost-effective oral health products which can prevent or treat dental caries.

Due to the human body's need for essential fatty acids that can be found in fish oil, the fatty acids are widely used in functional foods. Our recent discovery of omega-3 and omega-6 fatty acids can be utilized for their anti-microbial activity against cariogenic pathogens. Currently, no combinations of the fatty acids have been used to prevent or treat caries disease. There is a commercial opportunity for such products and there is no prior use of the fatty acid combinations as anti-caries treatment and prevention.

The present invention uses the novel anti-microbial activity and provide compositions of omega-3, omega-6, and other fatty acids for their synergistic effect. The current compositions provide novel recipes which could meet the market needs for anti-caries products.

### SUMMARY OF THE INVENTION

The purpose of the present invention is to provide a novel application of omega-3, omega-6, and other fatty acids to ensure the full exploitation of the anti-microbial activity for human health services. Specifically, the fatty acids found in fish oil or algae or plants, have significant anti-microbial activity, allowing their mixture to be applied to the prevention and treatment of caries disease. The application and composition is a novel use of fatty acids for the prevention and treatment of dental caries by using the synergistic effect of fatty acids.

The invention reveals the composition of omega-3 fatty acids (EPA and ALA) and omega-6 fatty acid (GLA), and other fatty acids, and the application for prevention and treatment of dental caries. The characteristics of the above methods and compounds are:
(1) Common omega-3 fatty acids (EPA and ALA) and omega-6 fatty acid (GLA), and hexanoic acid, myristic acid, palmitic acid, and palmitoleic acid, including their fatty acid methyl esters, and fatty acid ethyl esters have anti-microbial activity; novel anti-microbial activity against oral pathogens. Through research, the inventors found that, the omega-3 fatty acids (EPA, ALA), omega-6 fatty acid (ARA, GLA), hexanoic acid, myristic acid, palmitic acid, palmitoleic acid, and their fatty acid methyl esters, their fatty acid ethyl esters have strong anti-microbial bioactivity. The biological activity has a strong inhibitory effect on various oral pathogens, particularly Streptococcus mutans, but not limited.
(2) Fish oil/omega-3 and omeg-6 fatty acids, fish oil fatty acid methyl ester, and fish oil fatty acid ethyl ester are to be used in prevention and treatment of oral diseases. The present invention provides an application for oral disease prevention and control by omega-3 and omega-6 fatty acids, their fatty acid methyl esters and ethyl esters. The method for prevention and control of oral infections mentioned above is the composition of omega-3, omega-6, other fatty acids, their and fatty acid methyl esters, and also their fatty acid ethyl esters are prepared for oral health care, 0.01-20% of the content to the final product weight, where the specific content items based on the type of system and requirements.

The inventor verified the test by dissolving 1 mg of omega-3 fatty acids (EPA, and ALA) and omega-6 fatty acids (GLA), omega-3 fatty acid methyl esters (EPA-ME, ALA-ME), and omega-3 fatty acid ethyl esters (EPA-EE, ALA-EE) in 1000 μl ethanol solution; took 1 μl (final concentration 1 μg/ml) and 5 μl (final concentration 5 μg/ml) solution by adding 100 μl of Streptococcus mutans in the culture medium cultured overnight at 37° C. After the overnight culture, the medium was diluted 1:10000 times, 5 μl of the diluted culture was applied to blood agar plates and cultured overnight. The calculated number of colony forming units (CFU) on the plates were determined and compared with the control treatment to obtain the percentage of inhibition. It was found that the 1 μg/ml final concentration of omega-3 fatty acids, omega-3 fatty acid methyl esters, and omega-3 fatty acid ethyl esters could inhibit 50% of Streptococcus mutans growth. The compositions of these fatty acids have synergistic effect and have stronger anti-caries activity.

Omega-3 and omega-6 fatty acids, their fatty acid methyl esters and ethyl esters are biologically active components with strong anti-microbial role. The components will be used for the prevention and treatment of caries diseases.

Oral health care and therapeutic products include medicines, food, and daily necessities. Formulations of these products include the fish oil fatty acids, fish oil fatty acid methyl ester, fish oil fatty acid ethyl ester, additives and the appropriate form of the product carrier. In accordance with these products, they are prepared by conventional production methods.

The role of oral treatment with these drugs can be used in formulations such as tablets, sprays, powder, film, paint, ointment, gel, liquid, foam, mouthwash, etc. The treatments mainly refers to the tablets for oral medication in various form of tablets, including oral tablets, bio-adhesive patches, dispersible tablets, effervescent tablets, chewable tablets, tablets and other solutions. The fish oil fatty acids, fish oil fatty acid methyl ester, and fish oil fatty acid ethyl ester in the above-mentioned drugs should account for at least 0.1 percent to the weight of the mixture of drugs. The range of composition could be used in 0.01-20%.

The key role as mentioned above includes functional food such as chewing gum, beverages, toffee, and other dairy products. The fish oil fatty acids, fish oil fatty acid methyl ester, and fish oil fatty acid ethyl ester content should be at least 0.1% to weight mixture of food, although the range of composition could be used in 0.01-20%.

The key role as mentioned above includes oral daily necessities such as toothpaste, tooth powder, etc. The fish oil fatty acids, fish oil fatty acid methyl ester, and fish oil fatty acid ethyl ester content should be at least 0.1% to the weight of a mixture of daily necessities, although the range of composition could be used in 0.01-20%.

Provided by the invention of omega-3 fatty acids, omega-6 fatty acids, their fatty acid methyl esters, and ethyl esters having anti-microbial activity for the prevention and treatment of caries diseases, is the first time that compositions of mixed fatty acids were applied to prevention and treatment of dental caries.

### DETAILED DESCRIPTION OF THE INVENTION

The following examples are presented by way of illustration, not of limitation.

### Example 1

Dissolve 1 mg fatty acids, EPA, ALA, GLA, hexanoic acid, myristic acid, palmitic acid, and palmitoleic acid, in 1 ml ethanol to make a 1 mg/ml stock solution. Take 1 μl and 5 μl of above solutions, and mixed them up. And add the mixture into 100 μl Streptococcus mutans culture (at final concentration 1 μg/ml and 5 μg/ml), incubate the said bacterial culture overnight at 37° C. Afterwards, dilute the overnight culture by 1:10000. Five μl of the diluted culture mix was applied to blood agar plate and cultured overnight. Calculate the clone number on the plate and compared with the negative control plate. Final concentration of 1 μg/ml of fatty acid mixture could significantly inhibit the growth of Streptococcus mutans. The result showed that the IC₅₀ of the fatty acid mixture is less than 1 μg/ml.

### Example 2

Dissolve 1 mg fatty acid ethyl esters, EPA-EE, ALA-EE, GLA-EE, hexanoic acid-EE, myristic acid-EE, palmitic acid-EE, and palmitoleic acid-EE, in 1 ml ethanol. Take 1 μl and 5 μl of the above solutions and mix them up. And the mixture solutions were added into 100 μl of Streptococcus mutans culture (at final concentration 1 μg/ml and 5 μg/ml), and cultured overnight at 37° C. Dilute this culture by 1:10000. Five μl of diluted solution was applied to blood agar plate and cultured overnight. Calculate the clone number on the disk, and compared with the negative control. Results showed that fatty acid ethyl ester mixture has significant anti-bacterial activity. A final concentration of 1 μg/ml extract could inhibit the growth of Streptococcus mutans. The result showed that the fatty acid ethyl ester mixture IC₅₀ is less than 1 μg/ml.

### Example 3

Dissolve 1 mg of acid methyl esters, EPA-ME, ALA-ME, GLA-ME, hexanoic acid-ME, myristic acid-ME, palmitic acid-ME, and palmitoleic acid-ME, in 1 ml ethanol. Take 1 μl and 5 μl of the above solution and mix them up. Add 1 μl and 5 μl of the mixture solution into 100 μA Streptococcus mutans solution (at final concentration 1 μg/ml and 5 μg/ml), and then cultured overnight at 37° C. Dilute this culture by 1:10000 and apply 5 μl of diluted solution to blood agar plate, and cultured overnight. Calculate the clone number on the plate and compare with the negative control. Results showed that the omega-3 fatty acid methyl esters have significant anti-bacterial activity. A final concentration of 1 μg/ml fatty acid methyl ester mixture could inhibit the growth of Streptococcus mutans. The study showed that the fatty acid methyl ester mixture has IC₅₀ of less than 1 μg/ml.

### Example 4

The weight percentage composition of anti-microbial oral chewing tablet, containing fish oil fatty acids (EPA, ALA, GLA, hexanoic acid, myristic acid, palmitic acid, and palmitoleic acid)/or their fatty acid methyl ester/or their fatty acid ethyl ester, is as follows:

| | |
|---|---|
| The fatty acid/or fatty acid methyl ester/or | 1% |
| ethyl ester mixture | |
| Lo Han Kuo extract | 1% |
| Dextrin | 18.5% |
| Menthol | 0.5% |
| HPMC | 4% |
| Manesium stearate | 0.5% |
| Other ingredients | 80% |

### Example 5

The weight percentage composition of chewing gum, containing fish oil fatty acids (EPA, ALA, GLA, hexanoic acid, myristic acid, palmitic acid, and palmitoleic acid)/or their fatty acid methyl esters/or their fatty acid ethyl ester, is as follows:

| | |
|---|---|
| Fatty acids/or their methyl esters/or ethyl esters | 1-5% |
| Gum base | 80% |
| Xylitol, or sorbitol | 5% |
| Menthol | 0.2% |
| Lecithin | 0.6% |
| Edible flavors (such as banana or other flavors) | 0.9% |
| Edible citric acid | 0.2% |

### Example 6

The weight percentage composition of chewing gum, containing fish oil fatty acids (EPA, ALA, GLA, hexanoic acid, myristic acid, palmitic acid, and palmitoleic acid)/or fatty acid methyl esters/or fatty acid ethyl esters, is as follows:

| | |
|---|---|
| The fatty acids/or their methyl esters/or ethyl esters | 1-5% |
| Gum base | 80% |
| High intense sweetener mogroside (Lo Han Kuo extract), | 2% |
| or rebaudisoside | |
| Menthol | 0.2% |
| Lecithin | 0.6% |
| Edible flavors (such as banana or other flavors) | 0.9% |
| Edible citric acid | 0.2% |

### Example 7

The weight percentage composition of dairy products, containing omega-3 fatty acids (EPA, ALA, GLA, hexanoic acid, myristic acid, palmitic acid, and palmitoleic acid), or their fatty acid methyl ester/or fatty acid ethyl ester, is as follows:

| | |
|---|---|
| Fatty acid/or their methyl ester/or ethyl ester | 1-5% |
| Milk | 90-95% |
| Mannitol, or xylitol or sorbitol or syrup | 1-5% |
| Or high intense sweetener | 0.5~1% |

### Example 8

The weight percentage composition of candy, containing omega-3 fatty acids (EPA, ALA, GLA, hexanoic acid, myristic acid, palmitic acid, and palmitoleic acid), or their fatty acid methyl ester/or ethyl esters, is as follows:

| | |
|---|---|
| Fatty acid/or methyl ester/or ethyl ester | 1-5% |
| Milk powder | 5% |
| Mannitol, or xylitol or sorbitol | 1% |
| Dry starch syrup solid objects | 30~35% |
| Non-fat milk solids | 5~10% |
| Plant fat | 15-20% |
| Salt | 0.2-0.3% |
| Colloidal | 1.5-2.0% |

### Example 9

The weight percentage composition of toothpaste, containing omega fatty acids (EPA, ALA, GLA, hexanoic acid, myristic acid, palmitic acid, and palmitoleic acid)/or omega fatty acid methyl ester/or fatty acid ethyl ester, is as follows:

| | |
|---|---|
| Fatty acids/or their fatty acid methyl ester/ | 1-5% |
| or ethyl esters | |
| Baking soda | 35% |
| Xylitol | 5% |
| hydrogen peroxide | 5~10% |
| Plant oil | 15-20% |
| Flavors | 1.5-2.0% |

### Example 10

Take fatty acids (EPA, ALA, GLA, hexanoic acid, myristic acid, palmitic acid, and palmitoleic acid)/or their methyl ester/or ethyl ester dissolved in 20-50% ethanol aqueous solution, xylitol, sorbitol, or mogroside 1%, to make mouthwash. Conventional method is applied for preparation of the mouthwash production, where active content of fatty acids is about 0.1-10%. The mouthwash can be used as mouth rinse for dental caries.

### BEST MODE FOR CARRYING OUT THE INVENTION

The preferred embodiments of this invention are described herein, which includes the best mode that is known to the inventor for implementing the invention. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

### INDUSTRIAL APPLICABILITY

The anti-microbial compositions of this invention containing mixed omega polyunsaturated fatty acids and other fatty acids are capable of maintaining the original antimicrobial activity against Streptococcus mutans, and can be used in a variety of applications where antimicrobial activity is desired, such as in materials for consumer goods including oral health products, foods and beverages, functional foods, drugs, pharmaceuticals, dental/medical supplies, or in industrial products. Methods of preparing such compositions, and methods of using such compositions in the treatment and/or prevention of dental caries disease,

1. The present invention relates to compositions of omega-3 fatty acids, α-linolenic acid (ALA) and eicosapentanoic acid (EPA), omega-6 fatty acids (GLA), and, hexanoic acid (C6), myristic acid (C14), palmitoleic acid (C16) and palmitic acid (16), for anti-microbial activity against cariogenic pathogens. The composition of the fatty acids is used for the prevention and treatment of the infection leading to dental caries.

2. As mentioned in claim 1, the compositions of the fatty acids for the prevention and treatment of caries. The compositions are effective against cariogenic pathogens, for example, Streptococcus mutans and Lactobacillus fermentum.

3. As mentioned in claim 1, the application of α-linolenic acid (ALA) and eicosapentanoic acid (EPA), omega-6 fatty acids (GLA), and, hexanoic acid (C6), myristic acid (C14), palmitic acid (C16), and palmitoleic acid (C16), derived from fish oil or algae oil or plant oil, as well as from synthetic sources, in the prevention and treatment of dental caries infections.

4. The compositions of the above fatty acids are not limited to the free fatty acid forms, also including their fatty acid methyl esters and fatty acid ethyl esters.

5. The compositions of these fatty acids can be used, in combination of the vitamins A, C, E, D or K; and sweetened with Lo Han Kuo extract; but not limited to.

6. The role of the compositions can be used in any form of medicinal formulations, including chew, sprays, powder, gum, film, paint, ointment, gel, paste, liquid, foam, gel, chewable/chewing tablets, chewable/chewing gum, tablets and other solutions. The omega fatty acids are used at least for the content of 0.01 percent to the weight of the mixture of drugs, generally 0.01-10% better, but not limited to.

7. The role of the compositions can be used in any form of food formulations, including chewing gum, beverages, baby formula, and other dairy products, the above-mentioned compositions of omega-3, omega-6, and other fatty acids, in the above-mentioned content of food for at least 0.01% to weight mixture of food, generally 0.01-10% better, but not limited to.

8. The role of the compositions can be used in any form of the oral daily necessities, including toothpaste, tooth powder, mouthwash; the above-mentioned compositions of omega-3, omega-6, and other fatty acids, in the daily necessities for the content of at least 0.01% to the weight of a mixture of daily necessities, generally 0.01-20% better, but not limited to.
